(19) United States
(12) Patent Application Publication
(10) Pub. No.: US 2026/0345146 A1
(43) Pub. Date: Nov. 5, 2026

(54) TISSUE TRACTION DEVICES, SYSTEMS, AND METHODS

(71) Applicant: Boston Scientific Scimed, Inc., Maple Grove, MN (US)

(72) Inventors: Nathan Thomas Cummings, Worcester, MA (US); Scott E. Brechbiel, Johnston, RI (US); Sean Powell, Holden, MA (US)

(73) Assignee: Boston Scientific Scimed, Inc., Maple Grove, MN (US)

(21) Appl. No.: 19/665,230

(22) Filed: May 1, 2026

Related U.S. Application Data
(60) Provisional application No. 63/798,946, filed on May 2, 2025.

(51) Int. Cl. A61B 17/02

(52) U.S. Cl. CPC A61B 17/0218; A61B 2017/0225

(57) ABSTRACT

A tissue traction device in the form of a gantry or scaffold. The material of the tissue traction device is resilient, such as a shape memory material, such that coupling of a portion of the deployed tissue traction device to target tissue deforms the tissue traction device, and the deformation creates a restoring force which applies traction to the target tissue. As the target tissue is cut with respect to surrounding tissue, the tissue traction device exerts a traction force on the target tissue to lift the target tissue.

### CROSS-REFERENCE TO RELATED APPLICATIONS

This application claims the benefit of priority under 35 U.S.C. § 119 to U.S. Provisional Application No. 63/798,946, filed on May 2, 2025, the entire disclosure of which is hereby incorporated by reference herein for all purposes.

### FIELD

The present disclosure relates generally to the field of medical devices, systems, and methods for applying traction to anatomical tissue. More particularly, the present disclosure relates to devices, systems, and methods for applying traction to anatomical tissue within a patient, such as within a body lumen within the patient.

### BACKGROUND

Minimally-invasive (e.g., transluminal, transcatheter, endoscopic, etc.) surgical techniques like endoscopic Submucosal Dissection (ESD) and endoscopic Mucosal Resection (EMR) typically allow for faster recovery than with open or laparoscopic surgical procedures. However, because such procedures are minimally invasive, there is limited space to maneuver within the body, and such procedures typically require a high degree of expertise. In procedures involving cutting of tissue, one of the largest time and complexity drivers is managing the tissue being cut. The loose section of tissue may obstruct visibility, such as by falling on the instruments being used, occluding visibility of the imaging device (e.g., camera), and creating a hindrance affecting movement of the instruments used during the procedure and in reaching all regions and depths of the target tissue being cut. Various solutions for lifting the cut (and often hanging) mass of tissue, thus clearing the path for visibility and operation of medical tools and devices, have been developed. However, positioning of the elements used with such solutions may be challenging, particularly in a space-restricted environment. Moreover, as tissue is cut, a continual increase in traction is required, presenting various challenges during the procedure. Additionally, the devices used with such solutions may require separate medical devices than those used to perform the procedure, and such devices may take up valuable space from the instruments performing the procedure. For instance, the use of tissue traction devices is advantageous in ESD procedures to apply traction to tissue and thereby facilitate visibility and help separate tissue planes. However, existing tissue traction solutions are not ideal: Multiple traction devices (e.g., tissue clips, traction bands, etc.) may be used to apply traction and/or traction devices may need to be adjusted during performance of a procedure. For instance, as each traction device is placed and tissue is cut, the traction device provides increasingly less tension as the tissue flap grows with cutting of the tissue, and the traction force must be adjusted by moving the traction device and/or deploying an additional traction device. As procedures such as endoscopic submucosal dissection continue to evolve with new techniques, and new instruments are designed for making the procedure faster and safer, there remains a need for improvements to application of traction to tissue during the ESD procedure. It is with respect to these and other considerations that the present improvements may be useful.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In some aspects, a tissue traction device configured for delivery and deployment within a body lumen of a patient adjacent target tissue on which a procedure is to be performed includes, in accordance with various principles of the present disclosure, an elongated member formed into a curved shape which shifts from a delivery configuration into a deployed three-dimensional curved configuration defining a gap in the shape. In some aspects, the curved shape is curved about a curvature axis to extend around the interior of the body lumen to seat against the body lumen; and the elongated member is resilient so that coupling of a portion of the three-dimensional curved shape to the target tissue deforms the elongated member to impart a traction force to the target tissue.

In some aspects, the tissue traction device is self-expanding from the delivery configuration into the deployed configuration.

In some aspects, the curved shape is a loop. In some aspects, the loop includes a narrowed intermediate portion defining a sub-loop on either side of the narrowed-intermediate portion. In some aspects, the narrowed intermediate portion is formed by crossing the elongated member over itself. In some aspects, the curved shape is configured to seat within a body lumen with the sub-loops extending on either side of the axis of the body lumen. In some aspects, the curved shape is configured to seat within a body lumen with the sub-loops extending along the axis of the body lumen. In some aspects, the curved shape is in the form of a figure-eight. In some aspects, the curved shape is in the form of an hourglass with flattened ends. In some aspects, the narrowed intermediate portion is configured to be grasped by a tissue-engagement element.

In some aspects, the curved shape is curved about a curvature axis into a C-shape, defining the gap between ends of the curved shape which approach each other.

In some aspects, a tissue traction system for applying traction to target tissue within a body lumen includes, in accordance with various principles of the present disclosure, a tissue traction device formed of an elongated member formed into a curved shape defining a gap in the shape, in which the elongated member is resilient so that coupling of a portion of the elongated member to the target tissue deforms the curved shape to impart a traction force to the target tissue; and a tissue-engagement element configured to grasp the tissue traction device and the target tissue to apply traction to the target tissue via the tissue traction device.

In some aspects, the system further includes a retainer configured to constrain the tissue traction device in a delivery configuration. In some aspects, the system further includes a delivery device configured to deliver the tissue traction device wrapped over the exterior of the delivery device, and the retainer is configured to constrain the tissue traction device wrapped around the exterior of the delivery device. In some aspects, the retainer is a delivery device with a lumen, the tissue traction device deliverable in a delivery configuration within the lumen.

In some aspects, a method of applying traction to target tissue within a patient’s body, includes, in accordance with various principles of the present disclosure, deploying a tissue traction device within a body lumen of the patient and adjacent the target tissue, the tissue traction device being formed of a resilient material and expanding into a configuration seating against and along the interior surface of the body lumen; positioning the target tissue within a gap formed by the tissue traction device; and coupling a portion of the tissue traction device to the target tissue and deforming a portion of the tissue traction device such that the resilient material of the tissue traction device imparts a traction force to the target tissue.

In some aspects, the method further includes deploying the tissue traction device within the body lumen to self-expand within the body lumen to be seated against the interior of the body lumen.

In some aspects, the method further includes comprising coupling a portion of the tissue traction device to the target tissue with a tissue-engagement element. In some aspects, the method further includes coupling the tissue-engagement element to a narrowed intermediate portion of the tissue traction device and to the target tissue. In some aspects, the tissue traction device and the target tissue are coupled by only the tissue-engagement element.

These and other features and advantages of the present disclosure will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1A is a perspective view of an example of an embodiment of a tissue traction device formed in accordance with various principles of the present disclosure.

FIG. 1B is an end view (along a longitudinal end of a deployment site) of the tissue traction device illustrated in FIG. 1A.

FIG. 1C is a top view of the tissue traction device illustrated in FIG. 1A.

FIG. 1D is a side view (along a side of the deployment site extending along a longitudinal axis of the deployment site) of the tissue traction device illustrated in FIG. 1A.

FIG. 2A is a perspective view of an example of an embodiment of a tissue traction device formed in accordance with various principles of the present disclosure.

FIG. 2B is an end view (along a longitudinal end of a deployment site) of the tissue traction device illustrated in FIG. 2A.

FIG. 2C is a top view of the tissue traction device illustrated in FIG. 2A.

FIG. 2D is a side view (along a side of the deployment site extending along a longitudinal axis of the deployment site) of the tissue traction device illustrated in FIG. 2A.

FIG. 3A is a perspective view of an example of an embodiment of a tissue traction device formed in accordance with various principles of the present disclosure.

FIG. 3B is an end view (along a longitudinal end of a deployment site) of the tissue traction device illustrated in FIG. 3A.

FIG. 3C is a top view of the tissue traction device illustrated in FIG. 3A.

FIG. 3D is a side view (along a side of the deployment site extending along a longitudinal axis of the deployment site) of the tissue traction device illustrated in FIG. 3A.

FIG. 4 is a view of an example of an embodiment of a tissue traction device, showing an example of an embodiment of an anti-migration feature thereof.

FIG. 5 illustrates an example of an embodiment of a tissue traction device in a delivery configuration.

FIG. 6 illustrates an example of an embodiment of a tissue traction device in a delivery configuration.

FIG. 7A illustrates an example of an embodiment of a tissue traction device deployed with respect to target tissue at a schematically illustrated deployment site.

FIG. 7B illustrates an end view of an example of an embodiment of a tissue traction device such as in FIG. 7A engaged with schematically illustrated target tissue.

FIG. 7C illustrates a perspective view of an example of an embodiment of a tissue traction device similar to that of FIG. 7A engaged with schematically illustrated target tissue, and illustrating an optional slide feature in a detail view.

FIG. 7D illustrates a perspective view of an example of an embodiment of a tissue traction device such as in FIG. 7A engaged with and applying traction to schematically illustrated target tissue.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

There has been a growing interest in the medical field in minimally-invasive procedures, e.g., transcatheter, endoscopic, laparoscopic, etc., which do not require open surgery (cutting open the patient), but, instead, access a target site within the patient via a natural orifice (or, in some instances, a small incision not considered to constitute an open-surgery cut). For the sake of convenience, and without intent to limit, such procedures may be broadly referenced herein as transluminal procedures, in contrast with surgical procedures utilizing large surgical openings made in the patient for generally direct access (e.g., without navigating through a device or internal body passage) to the target site at which a procedure is to be performed (e.g., target tissue). Various endoluminal surgical (ELS) procedures have been developed, such as, without limitation, Endoscopic Mucosal Resection (EMR), Endoscopic Submucosal Dissection (ESD), to be performed within a patient and not in direct view of the medical professionals performing the procedure. The lack of direct visibility, as well as typical limited space for maneuvering of medical instruments presents various challenges to the medical professionals performing the procedure.

Although EMR can be performed in piecemeal technique, pathology on tissue fragments is often challenging and patients require more frequent follow-up endoscopies due to higher rate of incomplete resections and recurrences. While EMR is associated with relatively low technical complexity, shorter procedure times, and a low risk of adverse events, en bloc EMR is limited typically to lesions which are less than 2.0 cm in diameter / width. More specifically, for large sessile or laterally spreading polyps, en bloc resection with convention EMR is nearly impossible for lesions greater than 2 cm. For colorectal lesions of greater than 2 cm, en bloc resection can only be achieved in about 30 % of cases.

Medical professionals thus often turn to ESD for resection of challenging lesions, as well as large lesions. It is not uncommon for ESD to be utilized to remove neoplasms between 2 - 7 cm. The European Society of Gastrointestinal Endoscopy (ESGE) recommends ESD be used first line in the removal of Gastric Superficial neoplastic lesions with very low risk of lymph node metastases (EMR may be considered if the lesion is less than 10 to 15 mm with a very low probability of advanced histology). Often, ESD is also recommended first line for superficial esophageal squamous cell cancer (with EMR an option for lesions which are less than 10 mm). For colonic and rectal lesions, ESGE recommends removal of lesions with polypectomy or EMR. However, ESD can be considered if there is a high suspicion of limited submucosal invasion (indicated by depressed morphology and an irregular or non-granular surface pattern), and especially if the lesion is greater than 20 mm, or in lesions that otherwise cannot be optimally and radically removed by snare-based techniques (such as EMR techniques).

Although ESD has been in practice for two decades, ESD remains a very technically challenging procedure. In Japan, where ESD procedure is best established, the complication rates (including perforation, peritonitis, and bleeding) are 3.5 % for gastric ESD, 3.3 % for esophageal ESD, and 4.6 % for Colorectal ESD. Outside of Japan and China, EDS is practiced by a relatively small number of highly technically-skilled endoscopists and surgeons. Long procedure times and high risk of perforation are still significant challenges which have prevented broad physician adoption.

In accordance with various principles of the present disclosure, devices, systems, and methods are described which may be used in minimally-invasive procedures, such as ESD, to facilitate manipulation of an anatomical structure within the patient’s body. The anatomical structure may be anatomical tissue, and the device may be configured to move a portion or layer of tissue relative to another portion or layer of tissue, such as by application of a traction force. It will be appreciated that reference may be made herein simply to “tissue” instead of “anatomical tissue” or “anatomical structure” for the sake of convenience and without intent to limit. In some aspects, the present disclosure is directed to devices, systems, and methods for moving, such as to separate and/or lift and/or apply traction to tissue within a patient’s body.

Traction is a foundational technique utilized in open and laparoscopic surgery. Traction may be used to move tissue or other anatomical structures and may be advantageously used to greatly enhance visualization of anatomy, tissue planes, and potential hazards such as nerves or blood vessels. Additionally or alternatively, traction may be used to impart a tension force to the tissue, which may change the way the tissue behaves as it is being cut or dissected. For instance, traction may be used in surgical procedures to “present” the tissue to be cut to the cutting instrument in an orientation or manner that makes the cutting easier and/or helps to ensure that the cut is made through the desired tissue in the desired direction. In some aspects, traction alters the nature and/or properties of tissue in a manner which may facilitate performance of a procedure with respect to or in the vicinity of the tissue under traction. It will be appreciated that principles of the present disclosure are applicable to tissue which is cut as well as tissue or other anatomical structures which are not cut. For instance, principles of the present disclosure are applicable for providing traction and/or counter-traction during various medical procedures, including, without limitation, surgical procedures that involve cutting of tissue. The use of traction is a basic precept of surgery during cutting procedures, but should be appreciated as having broader applicability as well.

In addition to the procedural benefits of using traction during endoscopic procedures, there is a mounting body of evidence that devices aiding with traction during endoscopic submucosal dissection reduce the technical complexity of the procedure, and significantly lower the learning curve associated with trainees and/or new ESD procedure adopters.

To date, current traction solutions, both commercialized and physician made, do not adequately address true user needs. At best, commercially-available traction solutions leave significant room for improvement with regard to efficiency, effectiveness, flexibility, and ease of use. In open surgery, traction typically is applied using the non-dominant hand directly or through a surgical instrument (typically some kind of forceps). In laparoscopic surgery, traction is applied via tissue graspers introduced through one of the surgical ports, which provide access to the target body cavity or space. This can be done by the lead surgeon, assisting surgeon, or scrub nurse. In endoscopic surgery, however, multiple portals for accessing the target surgical site generally are not provided, and, typically, only the endoscope (or other access device) provides both visualization and access. Although there are some endoscopes with two working channels, or devices that can attach to the outside of the endoscope to provide a secondary working channel, all of these access channels are either integral to the endoscope or run parallel to it, and therefore are directionally constrained, and limited in the degrees of freedom of movement and control that can be achieved when maneuvering other devices to the target site.

In accordance with various principles of the present disclosure, a tissue traction device is configured to be deployed within a patient’s body to provide a point from which traction may be applied to target tissue within the patient at a target site. It will be appreciated that reference may be made to a target site, treatment site, anatomical site, delivery site, deployment site, implant / implantation site, site of implantation, etc., interchangeably and without intent to limit. In some aspects, target tissue is defined within the treatment site and with respect to which a procedure is to be performed. The target tissue may be, but is not necessarily limited to, a lesion. In some aspects, the tissue traction device is specifically configured to be deployed within a body lumen of a patient to apply traction to target tissue within the body lumen. For instance, the tissue traction device may be expandable to form a gantry or scaffold or other stable structure with a portion or structure configured to be positioned opposite target tissue and which may be coupled to the target tissue to apply traction to the target tissue. In some aspects, the tissue traction device is expandable from a delivery configuration, in which the tissue traction device may be in a compact and/or collapsed and/or constrained and/or compressed configuration, to a deployed configuration, in which the tissue traction device is shifted into an expanded and/or unconstrained and/or relaxed configuration. In some aspects, the tissue traction device is self-expanding. In some aspects, the tissue traction device is formed of a shape memory material to facilitate shifting from a delivery configuration to a deployed configuration. In some aspects, the tissue traction device is configured to apply a radially-outward force upon shifting (e.g., expanding) into a deployed configuration to be securely seated against tissue at the deployment site, such as by pressing into the tissue. For instance, a tissue traction device formed in accordance with various principles of the present disclosure may radially expand into secure engagement with (e.g., pressing against, without damaging) the inner surface / interior of a body lumen to securely seat the tissue traction device with respect to the body lumen. It will be appreciated that compliant tissue may substantially conform to the general shape of the tissue traction device. In some aspects, the tissue traction device expands to the full diameter of the body lumen.

In some aspects, the tissue traction device is resilient or otherwise formed to be capable of applying traction to tissue by virtue of its own material properties. For instance, use of a shape memory material to form a tissue traction device facilitates application of traction upon moving, bending, deforming, or otherwise shifting of a portion of the tissue traction device. It will be appreciated that terms such as portion, section, limited extent, structure, segment, etc., may be used interchangeably herein without intent to limit unless otherwise specified. The shape memory of the material gains potential energy as the device is deformed, etc., and the potential energy acts as a restoring force tending to return the tissue traction device to its relaxed, deployed configuration. Such restoring force may be utilized to apply traction to tissue operably coupled with the tissue traction device. Use of a separate traction device, such as an elongated traction element (e.g., traction band, tether, etc.), is optional and not necessarily required. A tissue-engagement element (e.g., a clip) may be extended from a portion of the tissue traction device to the target tissue, with deformation of the tissue traction device creating tension which applies traction to the target tissue. As the target tissue is cut (e.g., separated from surrounding tissue), the tissue traction device applies traction, pulling the grasped target tissue (engaged by the tissue traction device) away from tissue surrounding the incision and away from the instruments performing the procedure (e.g., away from the cutting instrument) and/or the medical scope or other instrument being used to view the procedure.

In some aspects, the tissue traction device is self-stabilizing, such as along the longitudinal extent of the body lumen. For instance, in some aspects, the tissue traction device is sized, shaped, configured, dimensioned, and/or positioned so that it is stable with respect to the deployment site along target tissue without the need for further actions, such as coupling the tissue traction device to tissue, after separation / delivery of the tissue traction device from a delivery device. In some aspects, the tissue traction device includes tissue engaging anti-migration features to further enhance stabilization of the tissue traction device with respect to the body lumen.

In some aspects, the tissue traction device curves about the curved interior of a body lumen (along the inner surface of the body lumen wall), yet leaves ready access to target tissue to which traction is to be applied (and with respect to which a procedure may be performed). In some aspects, the tissue traction device may thus be considered to have a partial-cylindrical shape. In some aspects, the tissue traction device, when viewed from a longitudinal end (e.g., when viewed from a medical scope positioned within the body lumen), is generally C-shaped. It will be appreciated that the orientation of the tissue traction device with respect to the body lumen may be altered from the above-described orientation, with the tissue traction device curving generally about an axis transverse to (e.g., obliquely extending across) the longitudinal axis of the body lumen. In some aspects, the tissue traction device is sized, shaped, configured, positioned, and/or dimensioned so that a gap is formed in the tissue traction device. The tissue traction device may be deployed with the target tissue positioned in the gap and thus accessible by medical instruments performing a procedure at the target site as well as by the tissue traction device for application of traction thereto. It will be appreciated that terms such as instruments, tools, devices, accessories, etc., may be usable interchangeably herein without intent to limit.

In some aspects, the tissue traction device is formed by an elongated member arranged generally in a loop with an intermediate region (between the longitudinal ends of a generally elongated loop) at which the elongated member is twisted, joined, crossed over itself, or otherwise forms a narrower, constricted region of the tissue traction device. In some aspects, the tissue traction device may be considered to have a figure-eight or hourglass shape, such as by forming the elongated member into a loop and twisting or otherwise crossing over or joining the elongated member along an intermediate region (of the loop formed by the elongated member). Upon deployment, the narrowed intermediate portion of the tissue traction device, which may be considered a constriction of the tissue traction device, may be positioned opposite (e.g., substantially diametrically across from) the target tissue, with the target tissue positioned in the gap between the ends of the tissue traction device (e.g., when curved, such as into a C shape, across the body lumen). In some aspects, the narrowed intermediate portion of the tissue traction device is the portion which is coupled with the target tissue to apply traction to the target tissue. In some aspects, a tissue-engagement element may be used to grasp the tissue traction device at the narrowed intermediate portion and also to grasp the target tissue to apply traction to the target tissue via the tissue traction device. In some aspects, the narrowed intermediate area may retain a position of the tissue-engagement element with respect to the tissue traction device (e.g., may inhibit sliding of the tissue-engagement element with respect to the tissue traction device).

In some aspects, the tissue traction device may expand from a reduced delivery configuration when deployed at the target tissue site. For instance, the tissue traction device may be delivered in a constrained configuration, such as wrapped around the exterior of a delivery device (e.g., a medical scope) which delivers instruments for performing a procedure with respect to target tissue, or otherwise constrained with respect to the delivery device (e.g., constrained within a lumen of the delivery device, such as a working channel of a medical scope).

Various embodiments of tissue traction devices, systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiments are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified herein by common reference elements and, for the sake of brevity and convenience, and without intent to limit, the descriptions of the common features are generally not repeated. For purposes of clarity, not all components having the same reference number are numbered in the accompanying drawings. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 100 are generally designated with the same reference numbers increased by a multiple of 100 and redundant description is generally omitted for the sake of brevity. Moreover, certain features in one embodiment may be used across different embodiments and are not necessarily individually labeled when appearing in different embodiments. Finally, references to side and end views are with respect to the target site in which the tissue traction device described herein is positioned.

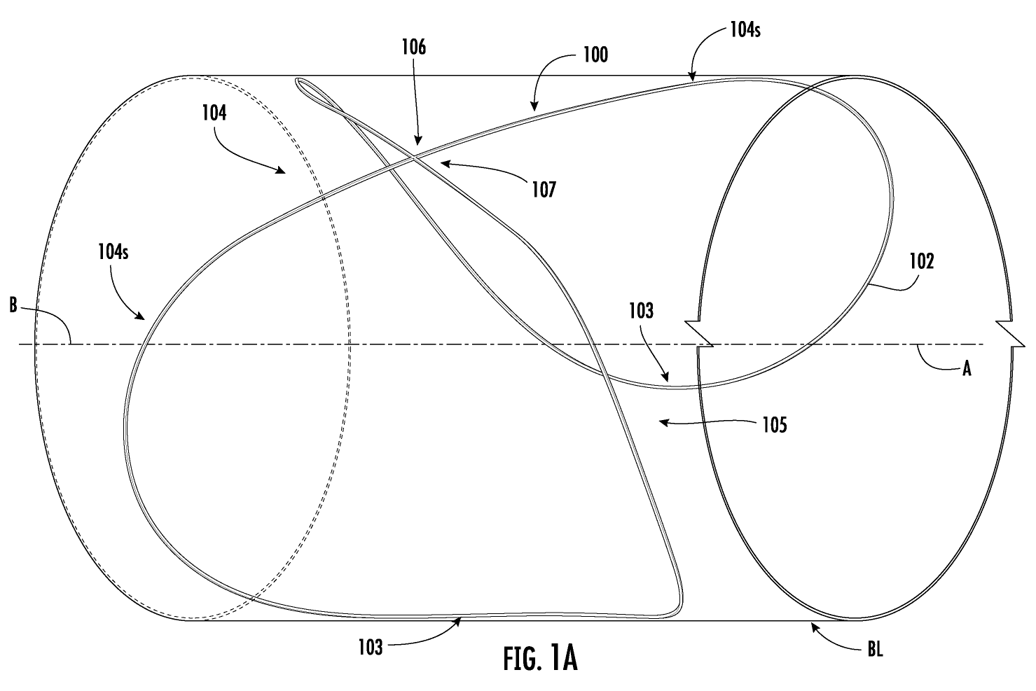

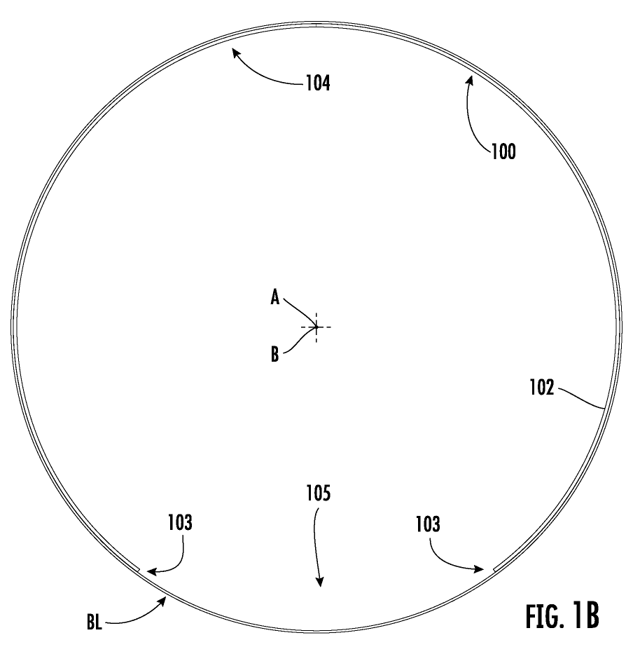

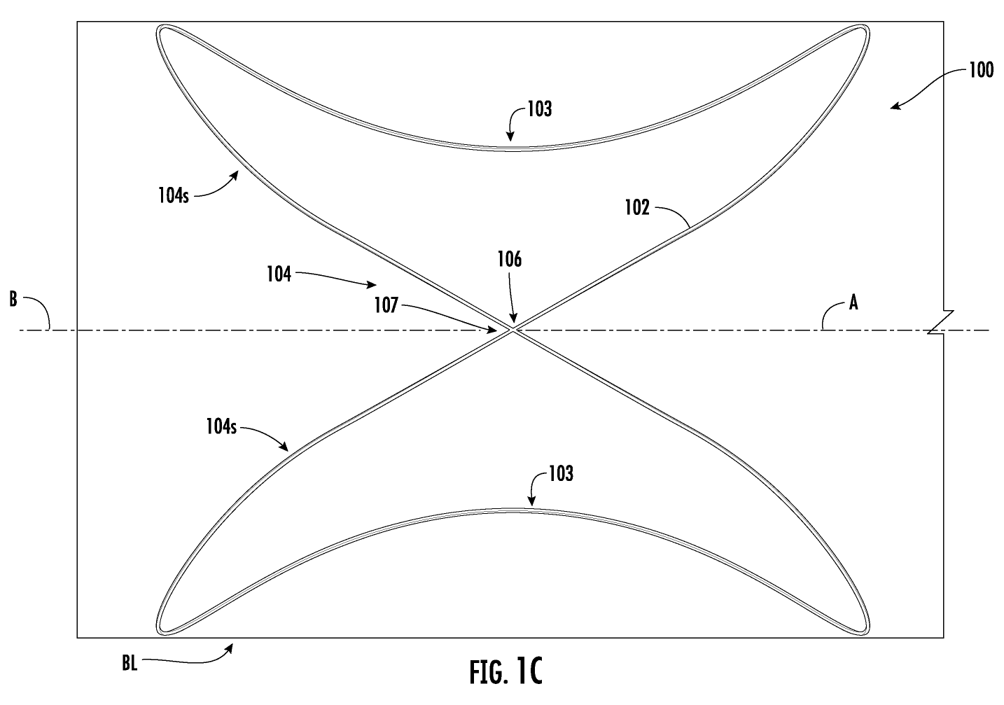

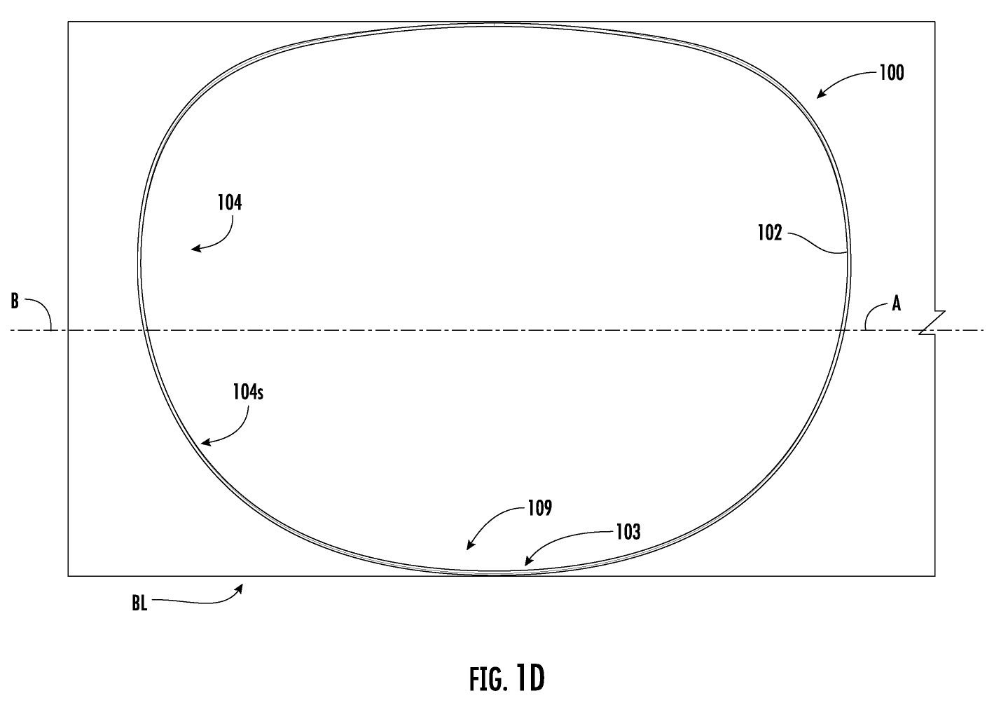

Turning now to the drawings, an example of an embodiment of a tissue traction device 100 illustrated in FIGS. 1A-1D is formed, in accordance with various principles of the present disclosure, from an elongated member 102 which is arranged in a loop 104 curved to be deployed within a body lumen. The elongated member 102 may be formed from a shape memory material set to retain a desired curved or otherwise bent configuration when unconstrained (e.g., in a relaxed configuration) and deployed within a patient’s body. More particularly, the shape memory material is selected to resume its intended, set shape when delivered to the target anatomy, and thus has a transition temperature corresponding to typical human or mammalian body temperature. In some aspects, the shape memory material has various properties, such as flexibility, elasticity, resiliency, etc., to be delivered in a compact configuration and to expand into a deployed configuration, such as may be readily appreciated by those of ordinary skill in the art. In some aspects, the shape memory material allows the tissue traction device 100 to be self-expanding from a delivery configuration into an expanded three-dimensional deployed configuration. It will be appreciated that the diameter, length, stiffness, etc., of a tissue traction device formed in accordance with various principles of the present disclosure may be selected based on the anatomical structure of the deployment site to ensure secure, stable deployment to support use in applying traction to target tissue. In some aspects, the tissue traction device is configured to expand to the full diameter of a body lumen in which it is positioned to be stabilized with respect to the interior wall of the body lumen (e.g., against the tissue along which the target tissue is located, as well as against tissue generally opposite, such as diametrically opposite, the target tissue). In some aspects, a larger device may be deployed within a body cavity, such as within a stomach, with the tissue traction device being stabilized by other features, and not necessarily expanding to the full size of the cavity in which the tissue traction device is deployed. Although examples of embodiments of tissue traction devices formed in accordance with various principles of the present disclosure are illustrated as deployed within a schematically illustrated body lumen, it will be appreciated that such deployment site is merely a non-limiting example of a deployment site.

As may be appreciated from the perspective view illustrated in FIG. 1A, and the end view illustrated in FIG. 1B (in a direction extending along the axis B of a schematically-illustrated body lumen BL), the loop 104 of the elongated member 102 is curved, about a curvature axis A. In accordance with various principles of the present disclosure, the loop 104 is curved to extend along, and generally in contact with a curved inner / interior wall of a body lumen BL, illustrated schematically in FIGS. 1A-1D. As such, when the tissue traction device 100 is delivered within a body lumen BL, with the curvature axis A substantially parallel to the body lumen axis B, the tissue traction device 100 may expand from a delivery configuration (a generally compact and/or compressed and/or constrained and/or collapsed configuration, such as described in further detail below) into engagement with the interior of a body lumen BL to form a scaffold or gantry (such terms being usable interchangeably herein without intent to limit). In some aspects, the loop 104 is curved into a partially cylindrical or C-shape when in an unconstrained configuration and viewed from an end thereof, with ends 103 approaching each other and leaving a gap 105 between the ends 103. As may be appreciated with reference to FIGS. 1A-1D, the ends 103 extend generally along the curvature axis A of the tissue traction device 100 and are spaced apart from each other along a direction transverse (e.g., oblique, such as orthogonal) to the curvature axis A and/or axis B of the body lumen BL to define the gap 105 between the ends 103 of the tissue traction device 100.

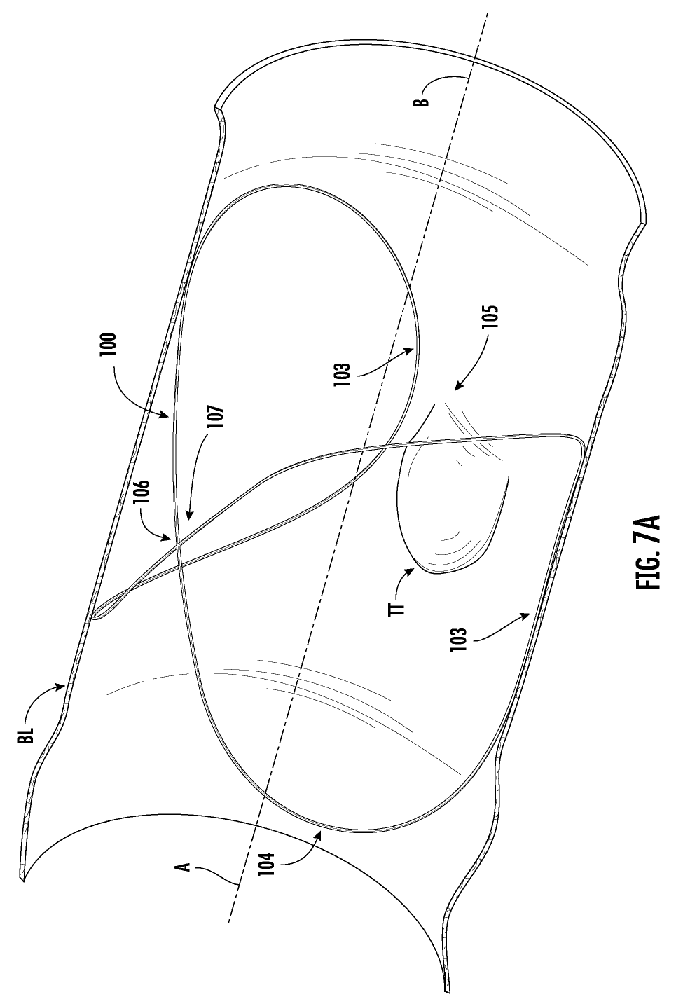

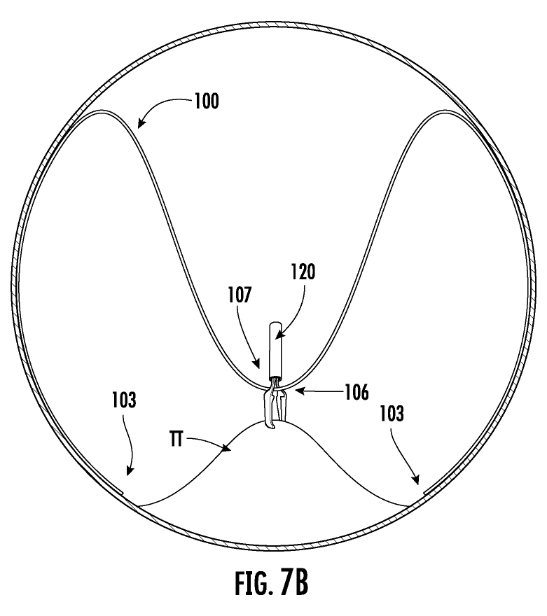

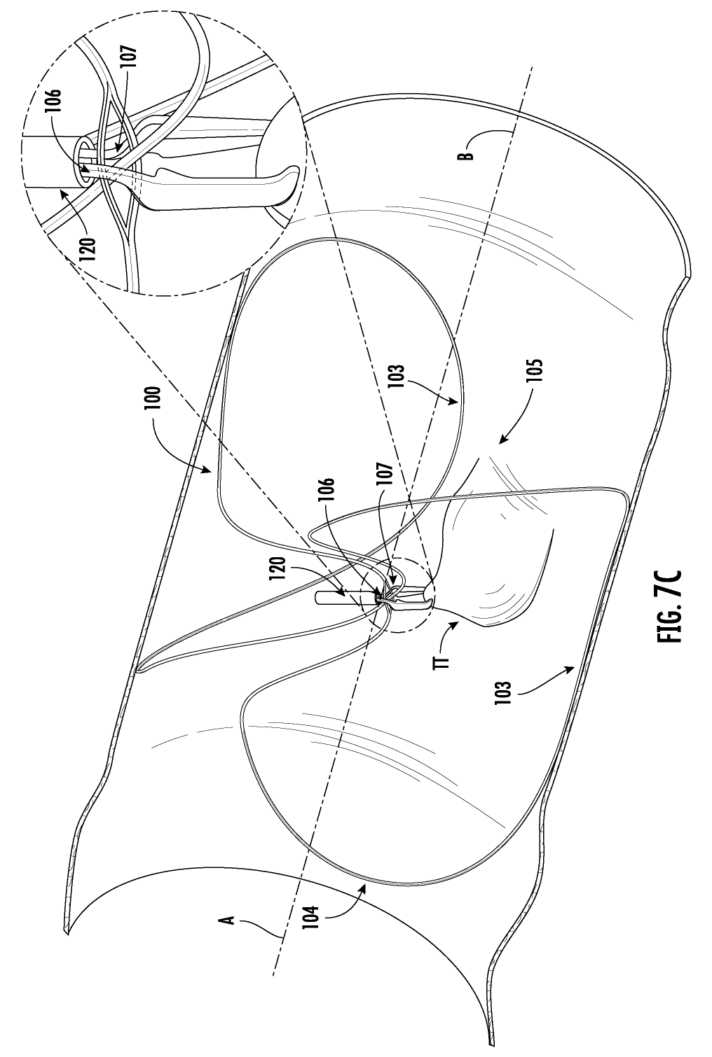

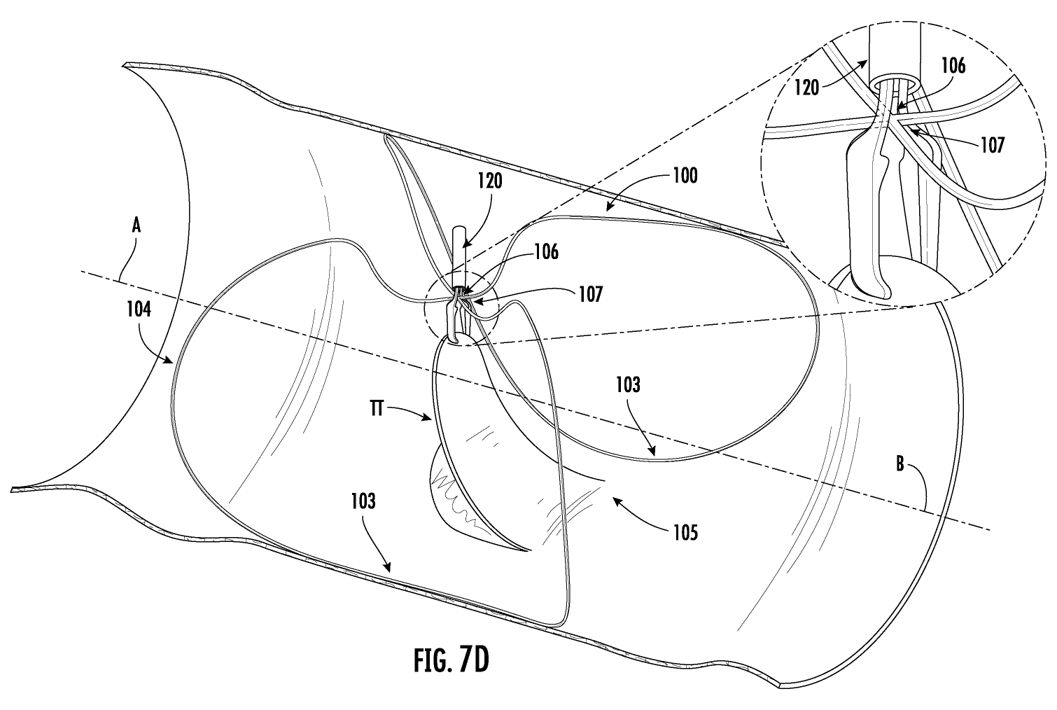

Various aspects of the tissue traction device 100, such as the shape memory properties, the size, shape, dimension, proportions, etc., contribute to secure deployment of the tissue traction device 100 at a deployment site. For instance, when deployed with respect to a body lumen BL, the tissue traction device 100 may be sized, shaped, configured, and/or dimensioned to expand to press against the interior of the body lumen BL to be securely deployed to resist movement with respect to the body lumen BL, without damaging the body lumen BL. It will be appreciated that those of ordinary skill in the art may readily understand the selection of such aspects or properties of the tissue traction device 100, such as based on the intended deployment site (e.g., properties of the body lumen BL such as size, elasticity, tissue wall type, etc.) such as to determine material selection, shaping, etc., of the tissue traction device 100. For instance, the length of a tissue traction device 100 of the present disclosure may be determined based on the deployment site, such as selection of a longer tissue traction device 100 for placement within a generally straight deployment site, versus a shorter tissue traction device 100 for placement within a nonlinear / curved deployment site. Once the tissue traction device 100 is deployed, a portion of the tissue traction device 100 may then be used to apply traction to target tissue TT within the body lumen BL, such as with the use of a tissue-engagement element 120 coupling the tissue traction device 100 and the target tissue TT, such as illustrated in FIGS. 7B-7D, and described in further detail below.

In some aspects, the curved configuration of the loop 104 of the elongated member 102 curves around a device curvature axis A from one end 103 to another end 103 of the tissue traction device 100. It will be appreciated that because the loop 104 is curved, ends 103 of the loop 104 may not be distinctly differentiated (e.g., with respect to surrounding extents of the loop 104, such as by a corner or other demarcation), but are generally opposite regions of the tissue traction device 100 as the tissue traction device 100 curves around the curvature axis A. In some aspects, as noted above, the ends 103 may be spaced apart from each other (e.g., are spaced apart along the circumference of a curved path along which the tissue traction device 100 curves, such as along the interior of the body lumen BL in which the tissue traction device 100 is deployed), such as illustrated in the end view of FIG. 1B. A space or gap 105 between the ends 103 of the tissue traction device 100 allows for placement of the tissue traction device 100 within a body lumen BL with the curvature of the tissue traction device 100 extending along the inner curvature of the body lumen BL and with target tissue positioned in the gap 105, between spaced-apart ends 103 of the tissue traction device 100, and unimpeded by the tissue traction device 100, such as illustrated in FIGS. 7A-7D, and discussed in further detail below.

In some aspects, as may be appreciated with reference to the perspective view of FIG. 1A and the top view of FIG. 1C, the loop 104 formed by the elongated member 102 may have a constriction 106 along an intermediate region 107 thereof (between the ends 103). In some aspects, the loop 104 may be considered to be formed into two or more sub-loops 104s (with a sub-loop 104s on each side of the constriction 106). As such, the tissue traction device 100 may be considered to form a shape resembling the number “8” (e.g., may be a “figure-eight” shape) and/or an hourglass. The constriction 106 may be formed by any of a variety of manners, such as by twisting the loop 104 or otherwise crossing the elongated member 102 over itself to form a crossed-over section thereof, the crossing point forming the constriction 106. Additionally or alternatively, the loop 102 of the elongated member 102 may be otherwise drawn together along the intermediate region 107 thereof, such as by shape setting the shape memory material, or applying a connector (e.g., band, clip, clamp, tie, etc.) over opposite sides of the loop of the elongated member 102 along the intermediate region 107 to form the constriction 106. The constriction 106 facilitates grasping of a portion or region or section (such terms being usable interchangeably herein without intent to limit unless stated) of the tissue traction device 100 and target tissue so that the tissue traction device 100 may apply traction force to the target tissue, as discussed in further detail below with reference to FIGS. 7A-7D. In some aspects, the constriction 106 is not fixed along the elongated member 102 because the elongated member 102 simply crosses over itself without being held together at the cross-over. In some aspects, a slide feature, such as a ring, loop, split segment, etc., such as illustrated in the detail view of FIG. 7C, allows for sliding movement of the elongated member 102, allowing for adjustability of the constriction 106 (e.g., modification of the relative angle of the crossed portion, or the location of the constriction 106 along the extent of the elongated member 102, etc.). The slide feature may be integrally formed in a portion of the elongated member 102 (e.g., a split in the elongated member 102) or a separate structure (e.g., a ring or loop) operably coupled to the elongated member 102). Such a slide feature may allow retention of closeness of the crossed-over portion of the elongated member 102 while also allowing relative movement (e.g., sliding) of the crossed-over portions. In some aspects, such a slide feature allows adjustability of the relative angle between the crossed-over portions of the elongated member 102.

The expansion of the tissue traction device 100 to expand against and/or to curve around the interior of the body lumen BL may serve to stabilize the tissue traction device 100 with respect to the body lumen BL against at least lateral movement (e.g., radially with respect to the axis B of the body lumen BL). As may be appreciated with reference to the side view of the tissue traction device 100 illustrated in FIG. 1D (a view along a side wall of the body lumen BL and in a direction generally orthogonal to the axis B of the body lumen BL), in some aspects, the tissue traction device 100 has a sufficient extent along the axis B of the body lumen BL to stabilize the tissue traction device 100 against pitching movement with respect to the axis B (rotation or pivoting about an axis orthogonal to the axis B). In some aspects, to enhance stabilization, the shape of at least one or each of the sub-loops 104s is not necessarily circular. In some aspects, the shape of at least one or each of the sub-loops 104s (along the curvature axis A) is oblate. In some aspects, at least one or each of the sub-loops 104s may have a stabilizing region 109, configured to be engaged with tissue when the tissue traction device 100 is deployed, with a larger radius of curvature (relative to a radius of curvature along other portions of the sub-loop 104s) or even a flattened region, such as to stabilize the tissue traction device 100 along the axis B of the body lumen BL, and against pitching movement with respect to the axis B.

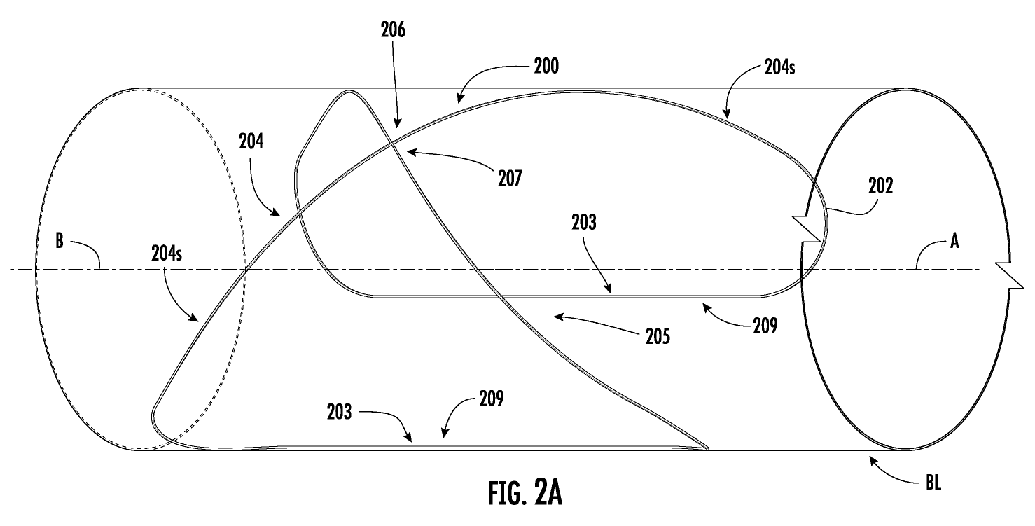

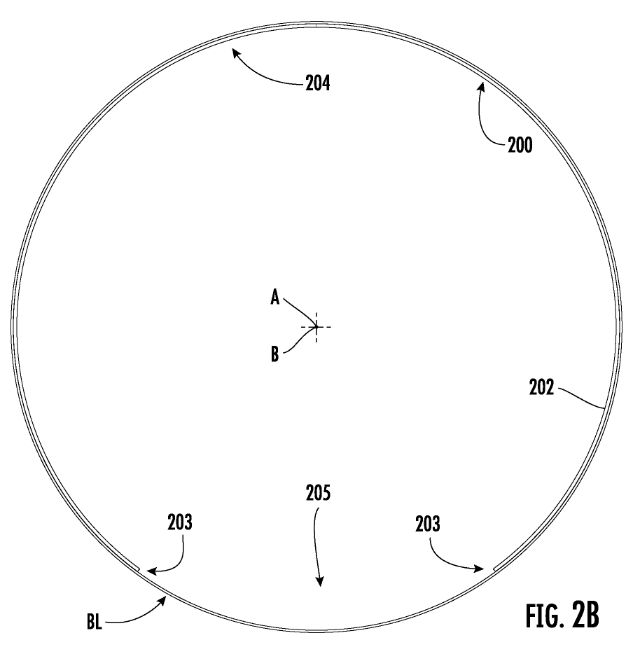

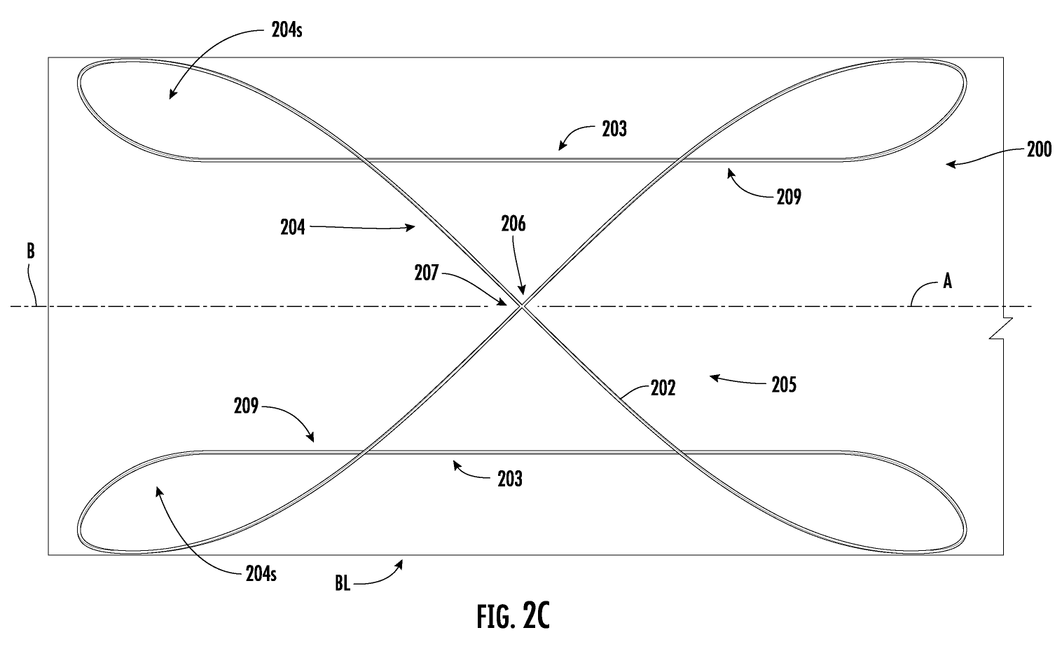

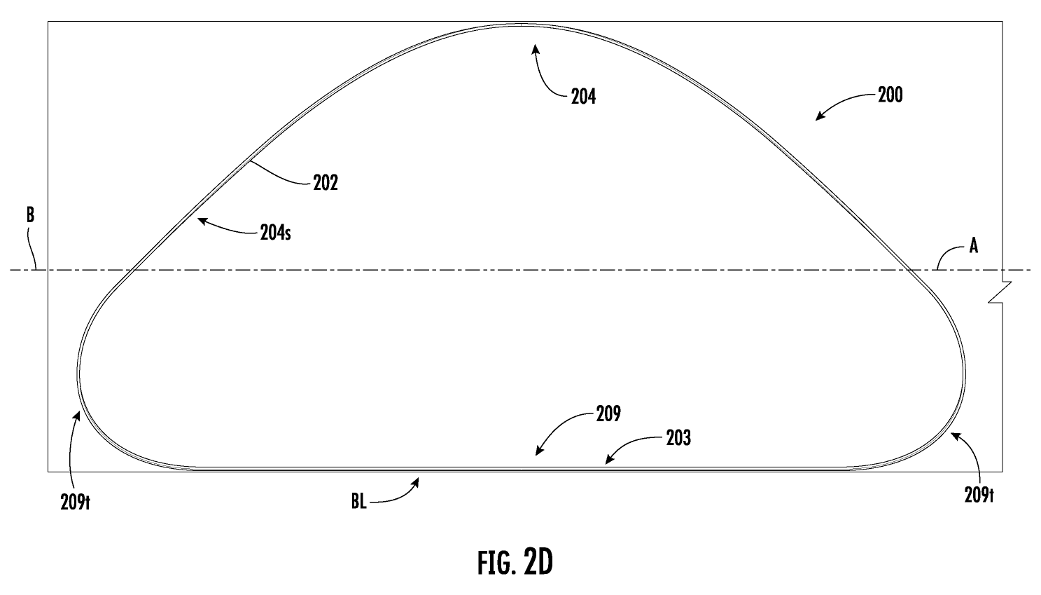

An example of an embodiment of a tissue traction device 200 with a more clearly defined stabilizing region 209, configured to stabilize the tissue traction device 200 against pitching movement with respect to the axis B (rotation or pivoting about an axis orthogonal to the axis B), is illustrated in FIGS. 2A-2D. As may be appreciated with reference to the perspective view of FIG. 2A, the top view of FIG. 2C, and the side view of FIG. 2D of the tissue traction device 200, the ends 203 of the tissue traction device 200 are relatively flattened (e.g., larger radius of curvature or substantially straight) compared to the more curved sections of the loop 204 of the elongated member 202 forming the tissue traction device 200 on either side of (e.g., immediately adjacent or contiguous with) the ends 203 of the tissue traction device 200. In some aspects, the loop 204 formed by the elongated member 202 forming the tissue traction device 200 forms a pair of generally triangular sub-loops 204s, as may be appreciated with reference to the perspective view of FIG. 2A, the top view of FIG. 2C, and the side view of FIG. 2D. In some aspects, as may be appreciated with reference to the side view of FIG. 2D, the elongated member 202 is configured to have a curved transition 209t to the generally flattened ends 203 so as to present a blunt, rather than a sharp, contour of the curved elongated member 202 to the tissue of the (schematically-illustrated) body lumen BL in which the tissue traction device 200 is deployed. It will be appreciated that the formation of ends 103 with a larger radius of curvature and/or a substantially straight extent may facilitate stable deployment of a tissue traction device 200 such as illustrated in FIGS. 2A-2D within a larger deployment site than a body lumen, such as within a cavity such as within a stomach. Such stabilization may be sufficient so that stabilization with respect to a body wall opposite the target tissue is optional and not necessary for stabilization of the deployed tissue traction device 200.

It will be appreciated that various other features and aspects of the tissue traction device 200 illustrated in FIGS. 2A-2D may be similar to corresponding features and aspects of the tissue traction device 100 illustrated in FIGS. 1A-1D, reference being made, for the sake of brevity and without intent to limit, to the above-descriptions of the gap 105, the constriction 106, and the intermediate region 107 of the tissue traction device 100 described above with reference to FIGS. 1A-1D as applying mutatis mutandis to gap 205, the constriction 206, and the intermediate region 207 of the tissue traction device 200 illustrated in FIGS. 2A-2D.

In some aspects, a tissue traction device formed in accordance with various principles of the present disclosure need not be positioned with its curvature axis (the axis about which the tissue traction device curves) oriented generally parallel to the longitudinal axis of the body lumen. Instead, it may be desirable for a curved tissue traction device formed in accordance with various principles of the present disclosure to be positioned within a body lumen with the curvature axis of the tissue traction device oblique to (e.g., generally orthogonal to) the longitudinal axis of the body lumen. In some aspects, such configuration and orientation of a tissue traction device of the present disclosure within a body lumen may enhance stabilization of the tissue traction device against “pitching” motion (pivoting about an axis oblique to the longitudinal axis of the body lumen).

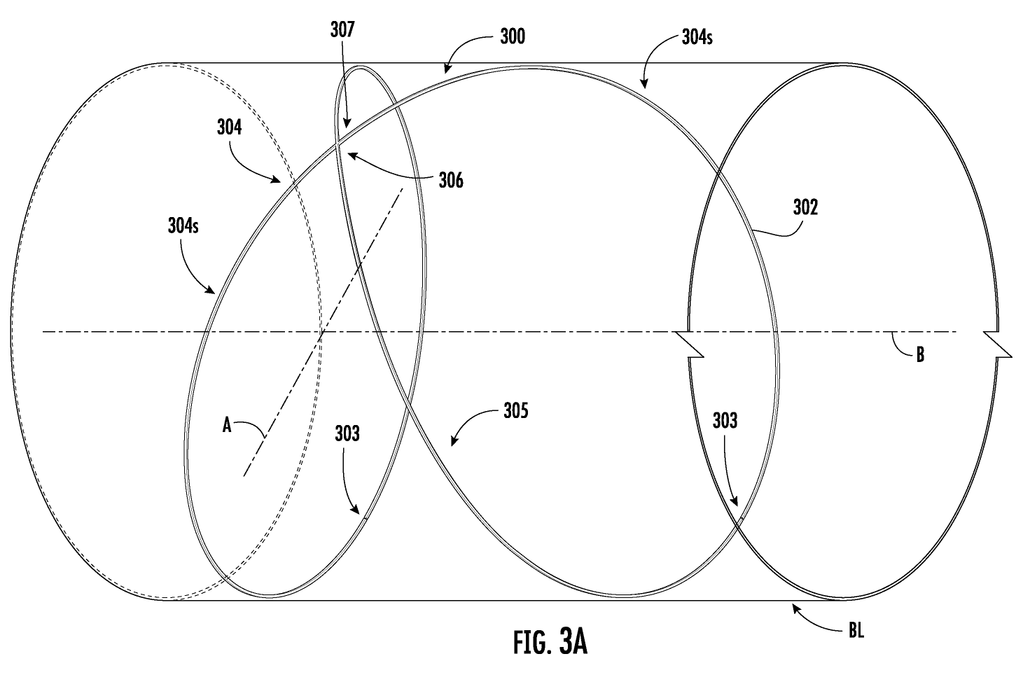

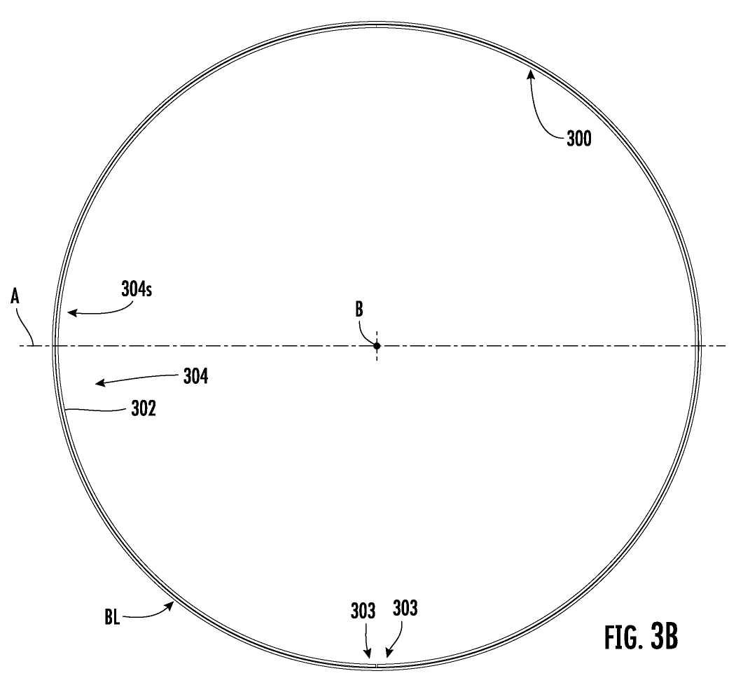

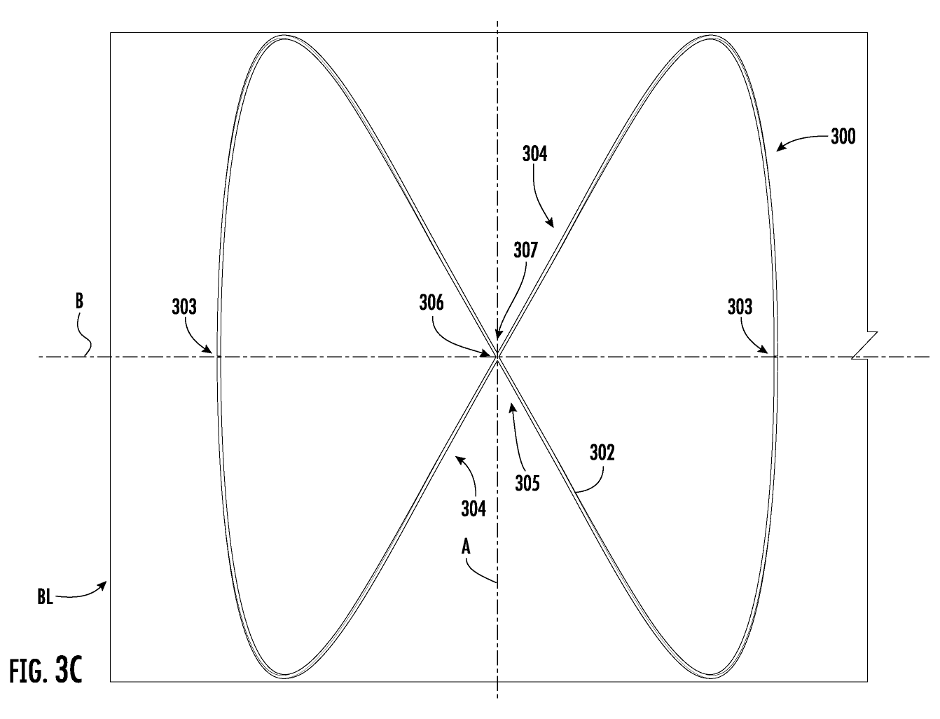

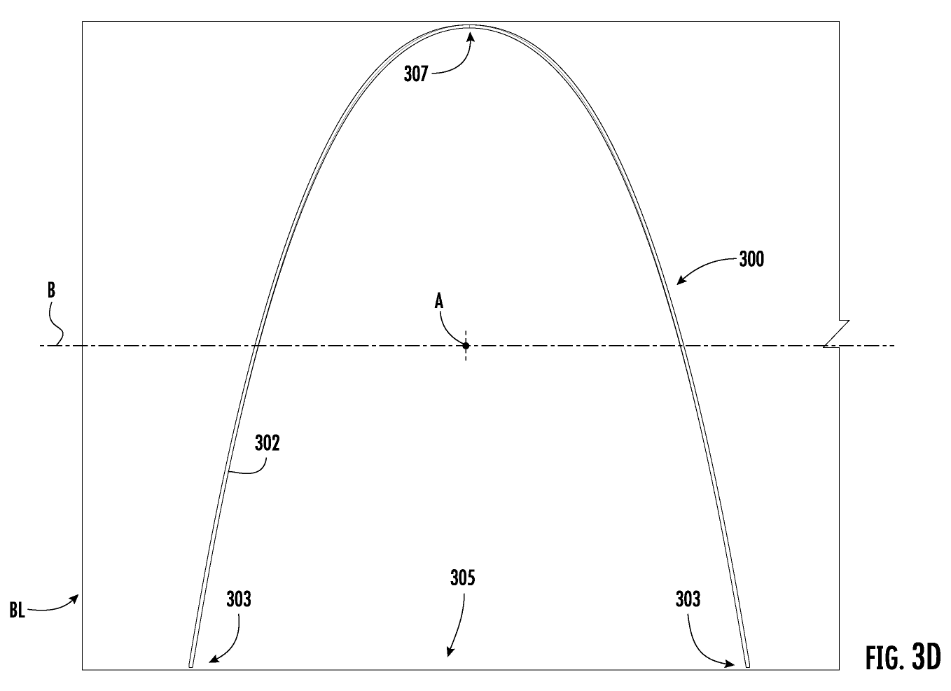

As illustrated in FIGS. 3A-3D, an embodiment of a tissue traction device 300 formed in accordance with various principles of the present disclosure is positioned within a schematically-illustrated body lumen BL with the curvature axis A of the tissue traction device 300 (the axis A about which the tissue traction device 300 curves) oblique to the axis B of the body lumen BL. The tissue traction device 300 is shaped and configured to be stabilized within the body lumen BL upon expansion into a relaxed, unconstrained configuration as illustrated in FIGS. 3A-3D. As may be appreciated with reference to the perspective view of FIG. 3A, the top view of FIG. 3C, and the side view of FIG. 3D, the ends 303 of the tissue traction device 300 are spaced apart from each other along the axis B of the body lumen BL. Such spacing of the ends 303 may stabilize the tissue traction device 300 within the body lumen BL. As may be appreciated with reference to the end view illustrated in FIG. 3B, each sub-loop 304s of the loop 304 of the elongated member 302 may be more circular than the sub-loops of the tissue traction device 100 of FIGS. 1A-1D such as to extend circumferentially around the inner concave curvature of the body lumen BL in which the tissue traction device 300 is deployed.

As may be appreciated from the top view of FIG. 3C, the loop 304 of the elongated member 302 forms a constriction 306 along an intermediate region 307 similar to the constrictions 106, 206 of the above-described examples of embodiments of tissue traction devices 100¸ 200 illustrated, respectively, in FIGS. 1A-1D and 2A-2D. The formation and/or use of the constriction 306 of the tissue traction device 300 illustrated in FIGS. 3A-3D may be substantially the same as the formation and/or use of the constriction 106, 206 of either or both of the tissue traction devices 100, 200¸ reference being made, for the sake of brevity and without intent to limit, to the above descriptions as applying mutatis mutandis to the tissue traction device 300 of FIGS. 3A-3D, with reference numbers the same as those used in FIGS. 1A-1D, but increased by 200.

Similar to the above-described examples of embodiments of tissue traction devices 100¸ 200 illustrated, respectively, in FIGS. 1A-1D and 2A-2D, the ends 303 of the example of an embodiment of a tissue traction device 300 illustrated in FIGS. 3A-3D may be spaced apart from each other (as may be appreciated with respect to FIGS. 3A, 3C, and 3D) to define a gap 305 so that the tissue traction device 300 may be deployed with target tissue positioned in the gap 305. However, in contrast with the above-described examples of embodiments of tissue traction devices 100, 200, the gap 305 extends from one end 303 of the tissue traction device 300 axially along the axis B of the body lumen BL to the other end 303 of the tissue traction device 300. To enhance stability of the tissue traction device 300 with respect to the body lumen BL, as may be appreciated from the side view illustrated in FIG. 3D, the ends 303 need not curve towards each other, such as in the tissue traction devices 100¸ 200. In some aspects, the curvature of the tissue traction device 300 along the curvature axis A may greater along the intermediate region 307 of the tissue traction device 300, and regions closer to (e.g., approaching) the ends 303 of the tissue traction device 300 may have a larger radius of curvature than the radius of curvature of the intermediate region 307, or may even be substantially straight / planar. In some aspects, the tissue traction device 300 is bent / curved along the intermediate region 307 to present a generally convex region of the tissue traction device 300 to the inner surface / interior of the body lumen BL, with reduced curvature in areas not necessarily contacting the body lumen BL. In some aspects, the tissue traction device 300 may be considered to be bent into an arch shape when viewed from the side (from a sidewall of the body lumen BL, and in a direction oblique or orthogonal to the axis B of the body lumen BL), such as illustrated in FIG. 3D.

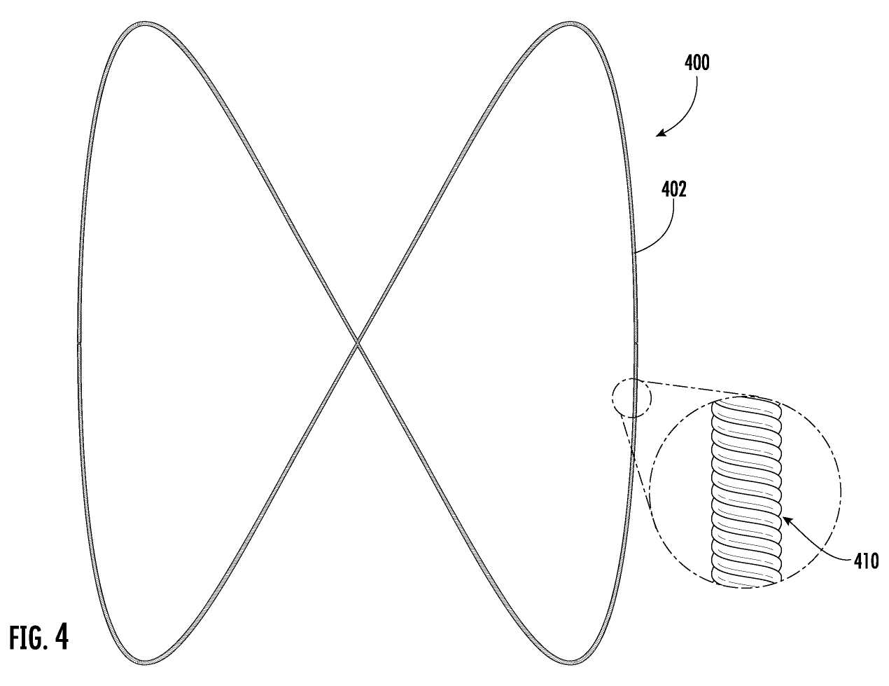

In some aspects, an elongated member forming a tissue traction device in accordance with various principles of the present disclosure may be formed to enhance stability with respect to the body lumen, such as with one or more anti-migration features. For instance, in the example of an embodiment illustrated in FIG. 4, anti-migration features 410 are provided along the elongated member 402 forming the tissue traction device 400. The anti-migration features 410 are sized, shaped, configured, and/or dimensioned to resist migration, slipping, or other undesired movement of the tissue traction device 400 with respect to a body lumen. For instance the anti-migration features 410 may be features / shapes which increase friction or other engagement with tissue of the body lumen, such as one or more of braids / twists / coils of a braided / twisted / coiled wire (such as illustrated in the detail view of FIG. 4), barbs, burrs, knurling, hooks, tines, pins, teeth (e.g., sheared and/or bent), prongs, surface-texturing, etc., whether on a single wire or a multi-component (e.g., braided or twisted) wire. In some aspects, the anti-migration features 410 are provided on at least a surface of the tissue traction device 400 which is positioned, upon deployment of the tissue traction device 400, to engage body tissue, yet which would not damage the lumen through which the tissue traction device 400 is delivered, such as in any of a variety of manners known to those of ordinary skill in the art. It will be appreciated that anti-migration features 410 may be applied to any of the tissue traction devices described herein or otherwise formed in accordance with various principles of the present disclosure.

In accordance with various principles of the present disclosure, tissue traction devices such as described herein may be configured for transluminal delivery into a patient. For instance, a tissue traction device formed in accordance with various principles of the present disclosure may be configured to be delivered into a patient via a natural orifice and/or small incision, and advanced and navigated within the patient through a passage (e.g., body lumen, and optionally within a lumen of a delivery device) to be deployed at a target site within the patient’s body. A tissue traction device formed in accordance with various principles of the present disclosure may thus be configured to be placed in a compact delivery configuration for delivery to the target site, and to be expandable upon deployment at the target site.

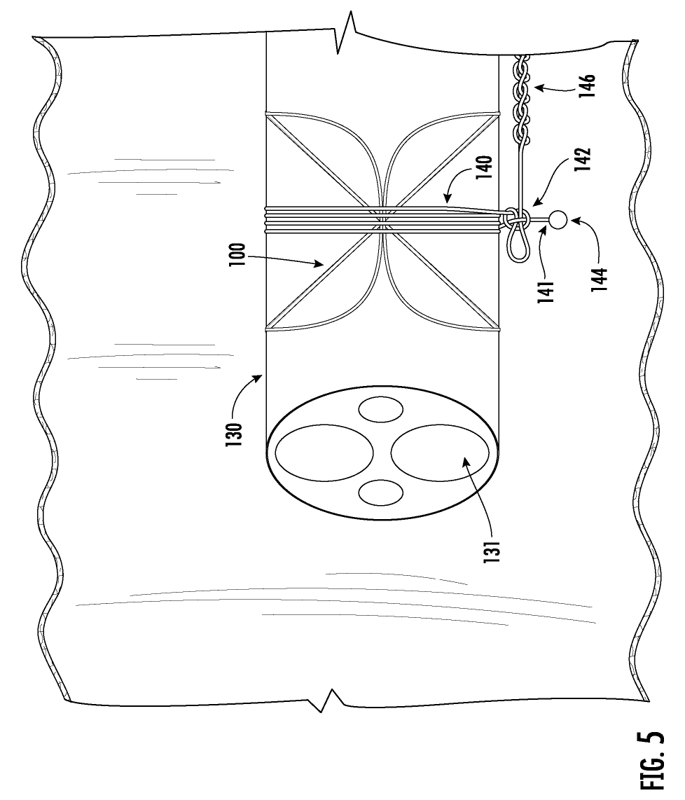

For instance, in some aspects, such as illustrated in FIG. 5, the tissue traction device 100 may be delivered by being wrapped around the exterior of a medical device 130 (e.g., a medical scope, a cap such as over a distal end of a medical scope, a catheter, a stylet, or any other appropriate, typically elongate, medical device such as any appropriate medical delivery device known to those of ordinary skill in the art). In some aspects, the medical device 130 may be considered a component of a tissue traction system which includes the tissue traction device, and optionally also the tissue-engagement element 120. The tissue traction device 100 may be retained in a delivery configuration (e.g., a constrained and/or compact and/or compressed and/or collapsed configuration) in any of a variety of manners. In the example of an embodiment illustrated in FIG. 5, a retainer 140 is applied over the tissue traction device 100 so that the tissue traction device 100 is retained in a delivery configuration. In some aspects, the retainer 140 is configured to be releasable by being actuated from outside the patient’s body. For instance, in the example of an embodiment illustrated in FIG. 5, the retainer 140 is in the form of a release string 140, such as any of a variety of elements (e.g., elongate elements) known to those of ordinary skill in the art, such as a string, a thread, a band, a cord, a wire, etc., which may be wrapped or otherwise configured to retain a delivery configuration of the tissue traction device 100. In some aspects, the release string 140 may extend proximally to be accessed by a medical professional, either directly, such as grasped by the medical professional’s hand, or via another actuator, such as an actuator coupled to the release string 140 and engageable and actuatable by the medical professional, such as, without limitation, manually. The release string 140 may extend proximally along the exterior of the medical device 130 or may be back-fed into a lumen 131 of the medical device 130 (e.g., the working channel of a medical scope over which the tissue traction device 100 is wrapped). A releasable knot 142 (such as, without, limitation, a mooring hitch knot) may retain the release string 140 in a retaining configuration around the exterior of the tissue traction device 100 and the medical device 130. In some aspects, a stopper 144 is provided on a free end 141 of the release string 140 so that the free end 141 of the release string 140 does not inadvertently pull through the releasable knot 142. In some aspects, the release string 140 is wrapped around the tissue traction device 100 two or more times (e.g., looped several times, such as two or more times, such as three, four, five, etc., times around the tissue traction device 100 in its delivery configuration). In some aspects, a chain of crochet loops 146, formed from the release string 140, may extend between adjacent wraps of the release string 140. Proximal pulling on the release string 140 would release the releasable knot 142 and optional crochet loops 146 to deploy the tissue traction device 100. It will be appreciated that provision of one or more (e.g., a chain of a plurality of) crochet loops 146 may increase the pull travel (extent or distance the release string 140 must be pulled) to release the release string 140 (e.g., untie the releasable knot 142). In some aspects, the crochet loops 146 reduce the risk of inadvertent release of the releasable knot 142 (e.g., the resultant longer pull travel is generally associated with intentional pulling to cause release of the releasable knot 142). In some aspects, the tissue traction device 100 may be released in an approximate location, and graspers or other instruments could be used to fine-tune the position of the tissue traction device 100 relative to the target tissue.

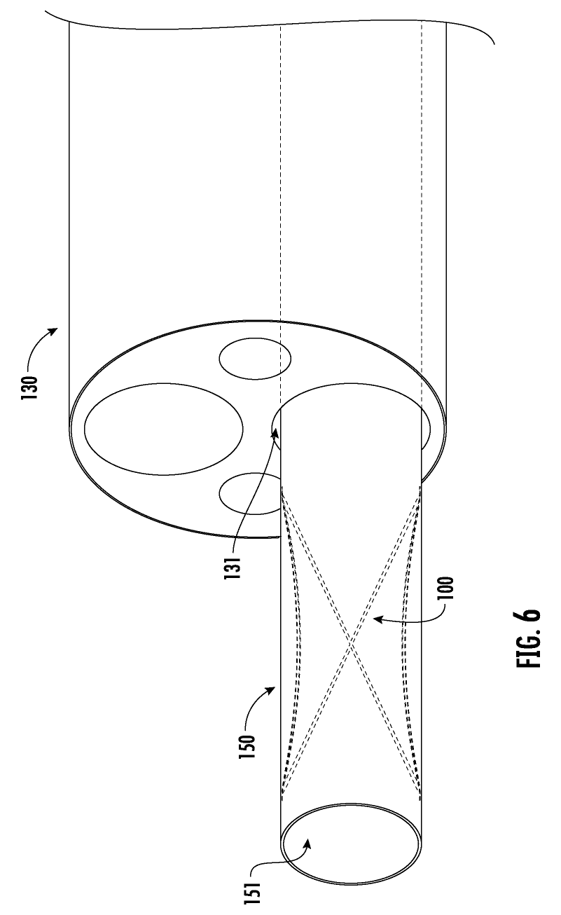

It will be appreciated that instead of being delivered over the exterior of a medical device 130, a tissue traction device formed in accordance with various principles of the present disclosure may be delivered within a lumen of a medical device. For instance, in the example of an embodiment illustrated in FIG. 6¸ an example of an embodiment of a tissue traction device 100 is delivered within a lumen 151 of a delivery device 150 which is extended through a lumen 131 of another delivery device, such as a medical device 130 as described above with reference to FIG. 5. In some aspects, the delivery device 150 may be considered a retainer which retains the tissue traction device 100 in a delivery configuration. As may be appreciated, any of a variety of deployment tools known to those of ordinary skill in the art may be used to deploy the tissue traction device 100 out of the lumen 151 of the delivery device 150, such as a pusher (e.g., an elongate element, such as known to those of ordinary skill in the art, positioned within the lumen 151 proximal to the tissue traction device 100). In some aspects, the delivery device 150 may be considered a component of a tissue traction system which also includes the tissue traction device, and optionally also the tissue-engagement element 120, and/or the medical device 130, and/or any of a variety of additional deployment tools such as known to those of ordinary skill in the art.

Once a tissue traction device formed in accordance with various principles of the present disclosure is deployed within a body lumen, the tissue traction device expands from its delivery configuration (e.g., constrained and/or compact and/or compressed and/or collapsed configuration) into a deployed configuration (e.g., unconstrained and/or expanded and/or relaxed configuration). In some aspects, such as noted above, the tissue traction device may be formed from a shape memory material or may otherwise be configured to be self-expanding (without the assistance of another device used to expand the tissue traction device) from the delivery configuration to the deployed configuration. An example of an embodiment of a tissue traction device 100 is illustrated in FIGS. 7A-7D as deployed within a body lumen BL within a patient’s body to apply traction to target tissue TT. As with reference to FIG. 4, it will be appreciated that although FIGS. 7A-7D illustrate a tissue traction device 100 such as illustrated in FIGS. 1A-1D (configured and deployed so that the curvature axis A of the tissue traction device 100 is substantially parallel to the axis B of the body lumen BL), any other configuration of a tissue traction device formed in accordance with various principles of the present disclosure may be depicted, instead, with substantially the same effect.

In use, an example of an embodiment of a tissue traction device 100 formed in accordance with various principles of the present disclosure may be deployed, as illustrated in FIG. 7A, with respect to target tissue TT within a body lumen BL within a patient’s body. As noted above, the tissue traction device 100 may expand upon deployment (e.g., be self-expanding) without the need for an additional device used to expand the tissue traction device 100. In some aspects, deployment and stabilization of the tissue traction device 100 with respect to the body lumen BL does not require further steps or devices to secure / anchor the tissue traction device 100 with respect to the body lumen BL. It will be appreciated that a tissue traction device formed in accordance with various principles of the present disclosure generally is conformable to the shape of the deployment site and/or the tissue at the deployment site will generally conform to the shape of the tissue traction device. The tissue traction device 100 is illustrated as placed with respect to the target tissue TT such that the target tissue TT is positioned in the gap 105 between the ends 103 of the tissue traction device 100. The target tissue TT is thus unimpeded by the tissue traction device 100 and is accessible (e.g., by other medical instruments, such as may be delivered with a delivery device, such as known to those of ordinary skill in the art) for performing a procedure with respect to the target tissue TT as well as accessible for application of traction thereto by the tissue traction device 100. As also illustrated in FIG. 7A, the tissue traction device 100 may be deployed with respect to the body lumen BL with the constriction 106 of the tissue traction device 100 (along the intermediate region 107 of the tissue traction device 100) generally opposite (e.g., substantially diametrically across from) the target tissue TT. For instance, the tissue traction device 100 in its deployed, expanded configuration is stabilized with respect to the body lumen BL (e.g., in contact and generally exerting a radially-outward force with respect to the interior of the body lumen BL) with the constriction 106 spaced apart (e.g., substantially diametrically across the body lumen BL) from the target tissue TT.

Once the tissue traction device 100 has been deployed, the constriction 106 may be operably associated with the target tissue TT to apply traction to the target tissue TT. In some aspects, a tissue-engagement element 120, such as, without limitation, a tissue clip (as illustrated), may be used to engage (e.g., grab, grasp, clasp, clip, etc.) the constriction 106 as well as to engage (e.g., grab, grasp, clasp, clip, etc.) the target tissue TT, such as illustrated in FIGS. 7B and 7C. It will be appreciated that, in some aspects, additional elements, such as an elongate traction element (e.g., a band, wire, tether, loop, etc.) may be used between the constriction 106 and the target tissue TT (e.g., between the constriction 106 and the tissue-engagement element 120). In some aspects, the tissue traction device 100 and the tissue-engagement element 120 (with one or more components thereof) may be considered to together form a tissue traction system. It will be appreciated that a tissue traction device 100 formed in accordance with various principles of the present disclosure may be used with only a single tissue-engagement element 120 without defects being made to non-target anatomy.

By pulling the constriction 106 of the tissue traction device 100 toward the target tissue TT and operably coupling the constriction 106 with the target tissue TT, such as illustrated in FIGS. 7B and 7C, the shape memory of the tissue traction device 100 applies a restoring force to the tissue traction device 100. The restoring force functions to return the tissue traction device 100 to a substantially unconstrained configuration, such as the deployed configuration of FIG. 7A. In other words, deformation of the tissue traction device 100, which may be appreciated with reference to FIGS. 7B and 7C, creates tension in the tissue traction device 100, pulling the constriction 106 of the tissue traction device 100 towards the initial position of the constriction 106, spaced apart from the target tissue TT. As the target tissue TT is cut, the tissue traction device 100 thus lifts the cut tissue away from surrounding tissue, as illustrated in FIG. 7D.

As may be appreciated, a tissue traction system of the present disclosure provides a tissue traction device which may be deployed and left in place out of the way of a target site at which a procedure is to be performed, yet available for applying traction to target tissue. As such, the delivery and deployment of a tissue traction device is simplified, with fewer instruments and steps than required by prior art devices and systems and method.

It will be appreciated that devices and systems of the present disclosure may be used for treating various anatomical tissue and/or anatomical systems, such as gastrointestinal, abdominal, digestive, urinary, reproductive, respiratory, cardiovascular, circulatory, etc. It will be appreciated that reference to a body passage includes naturally-existing passages (e.g., the colon) as well as medically-created passages (e.g., a passage created with the use of a medical instrument, and not existing without medical intervention) or otherwise. It will further be appreciated that the medical devices and systems described herein may be navigated within and through various body lumens, or other lumens, including, for example, catheters, ureteroscopes, bronchoscopes, colonoscopes, duodenoscopes, arthroscopes, cystoscopes, hysteroscopes, and the like. The disclosed medical devices and systems may also be inserted via different access points and approaches, e.g., percutaneously, endoscopically, laparoscopically, or combinations thereof.

It is to be understood by one of ordinary skill in the art that the present discussion is a description of illustrative examples of embodiments only, and is not intended as limiting the broader aspects of the present disclosure. All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. In particular, it will be clear to those skilled in the art that principles of the present disclosure may be embodied in other forms, structures, arrangements, proportions, and with other elements, materials, and components, without departing from the concept, spirit, or scope, or characteristics thereof. Other examples of manners of implementing the disclosed principles will occur to a person of ordinary skill in the art upon reading this disclosure. It should be apparent to those of ordinary skill in the art that variations and/or modifications, such as of structure, arrangement, proportions, materials, components, and otherwise, can be applied to the disclosed devices, systems, and/or methods, and/or to the sequence of steps of the method described herein without departing from the concept, spirit, and scope of the disclosure. For instance, variations or modifications may be made to adapt devices, systems, or methods of the present disclosure to specific environments and operative requirements without departing from the principles or spirit or scope of the present disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes, modifications, and/or additions apparent to those skilled in the art are deemed to be within the spirit, scope, and concept of the disclosure as defined by the appended claims. Various further benefits of the various aspects, features, components, and structures of tissue traction devices, systems, and methods such as described above, in addition to those discussed above, may be appreciated by those of ordinary skill in the art. The foregoing discussion has broad application and has been presented for purposes of illustration and description and is not intended to limit the disclosure to the form or forms disclosed herein.

The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader’s understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way

What is claimed is:

1. A tissue traction device configured for delivery and deployment within a body lumen of a patient adjacent target tissue on which a procedure is to be performed, said tissue traction device comprising:
an elongated member formed into a curved shape which shifts from a delivery configuration into a deployed three-dimensional curved configuration defining a gap in the shape;
wherein:
the curved shape is curved about a curvature axis to extend around the interior of the body lumen to seat against the body lumen; and
the elongated member is resilient so that coupling of a portion of the three-dimensional curved shape to the target tissue deforms the elongated member to impart a traction force to the target tissue.

2. The tissue traction device of claim 1, wherein said tissue traction device is self-expanding from the delivery configuration into the deployed configuration.

3. The tissue traction device of claim 1, wherein the curved shape is a loop.

4. The tissue traction device of claim 2, wherein the loop includes a narrowed intermediate portion defining a sub-loop on either side of the narrowed-intermediate portion.

5. The tissue traction device of claim 3, wherein the narrowed intermediate portion is formed by crossing the elongated member over itself.

6. The tissue traction device of claim 3, wherein the curved shape is configured to seat within a body lumen with the sub-loops extending on either side of the axis of the body lumen.

7. The tissue traction device of claim 3, wherein the curved shape is configured to seat within a body lumen with the sub-loops extending along the axis of the body lumen.

8. The tissue traction device of claim 3, wherein the curved shape is in the form of a figure-eight.

9. The tissue traction device of claim 3, wherein the curved shape is in the form of an hourglass with flattened ends.

10. The tissue traction device of claim 3, wherein the narrowed intermediate portion is configured to be grasped by a tissue-engagement element.

11. The tissue traction device of claim 1, wherein the curved shape is curved about a curvature axis into a C-shape, defining the gap between ends of the curved shape which approach each other.

12. A tissue traction system for applying traction to target tissue within a body lumen, said system comprising:
a tissue traction device formed of an elongated member formed into a curved shape defining a gap in the shape, wherein the elongated member is resilient so that coupling of a portion of the elongated member to the target tissue deforms the curved shape to impart a traction force to the target tissue; and
a tissue-engagement element configured to grasp the tissue traction device and the target tissue to apply traction to the target tissue via the tissue traction device.

13. The system of claim 12, further comprising a retainer configured to constrain said tissue traction device in a delivery configuration.

14. The system of claim 13, further comprising a delivery device configured to deliver said tissue traction device wrapped over the exterior of said delivery device, wherein said retainer is configured to constrain said tissue traction device wrapped around the exterior of said delivery device.

15. The system of claim 13, wherein said retainer is a delivery device with a lumen, said tissue traction device deliverable in a delivery configuration within the lumen.

16. A method of applying traction to target tissue within a patient’s body, said method comprising:
deploying a tissue traction device within a body lumen of the patient and adjacent the target tissue, wherein the tissue traction device is formed of a resilient material and expands into a configuration seating against and along the interior surface of the body lumen;
positioning the target tissue within a gap formed by the tissue traction device; and
coupling a portion of the tissue traction device to the target tissue and deforming a portion of the tissue traction device such that the resilient material of the tissue traction device imparts a traction force to the target tissue.

17. The method of claim 16, further comprising deploying the tissue traction device within the body lumen to self-expand within the body lumen to be seated against the interior of the body lumen.

18. The method of claim 16, further comprising coupling a portion of the tissue traction device to the target tissue with a tissue-engagement element.

19. The method of claim 18, further comprising coupling the tissue-engagement element to a narrowed intermediate portion of the tissue traction device and to the target tissue.

20. The method of claim 18, wherein the tissue traction device and the target tissue are coupled by only the tissue-engagement element.
